(19) United States
(12) Patent Application Publication
(10) Pub. No.: US 2017/0166859 A1
(43) Pub. Date: Jun. 15, 2017

(54) CELL CULTURE CARRIER MODULE, BIOREACTOR AND CELL RECOVERY METHOD

(71) Applicant: Industrial Technology Research Institute, Hsinchu (TW)

(72) Inventors: Hsiu-Ying Wang, New Taipei City (TW); Ying-Wen Shen, Miaoli County (TW); Ing-Kae Wang, Hsinchu City (TW); Shu-Fen Yeh, New Taipei City (TW); Wei-Zhou Yeh, Hsinchu City (TW)

(21) Appl. No.: 14/985,308

(22) Filed: Dec. 30, 2015

(30) Foreign Application Priority Data
Dec. 9, 2015 (TW) 104141270

(51) Int. Cl. C12M 1/00; C12M 3/00

(52) U.S. Cl. CPC C12M 47/02; C12M 23/44

(57) ABSTRACT

A cell culture carrier module, a bioreactor and a cell recovery method are provided. The cell culture carrier module includes at least one cell culture carrier capable of transforming between a two dimensional structure and a three dimensional structure. The cell culture carrier exhibits the two dimensional structure in a loosened state and exhibits the three dimensional structure in a compressed state.

### CROSS-REFERENCE TO RELATED APPLICATION

This application claims the priority benefit of Taiwan application serial no. 104141270, filed on Dec. 9, 2015. The entirety of the above-mentioned patent application is hereby incorporated by reference herein and made a part of this specification.

### BACKGROUND OF THE INVENTION

1. Field of the Invention

The disclosure relates to a cell culture carrier module, a bioreactor, and a cell recovery method. In particular, the disclosure relates to the cell culture carrier module able to transform between a two dimensional structure and a three dimensional structure, the bioreactor including the foregoing cell culture carrier module, and the cell recovery method utilizing the foregoing cell culture carrier module.

2. Description of Related Art

The commonly known carrier scaffold for cell mass production are divided into two categories, respectively represented by natural materials (for example, collagen, chitosan, gelatine, or the like) or synthetic materials (Polycaprolactone (PCL), Polystyrene (PS), Polypropylene (PP), Poly(lactic-co-glycolic acid) (PLGA), or the like). The natural materials are mostly materials derived from animal sources. Although the materials derived from animal sources have a lower cytotoxicity and a higher biocompatibility, the materials derived from animal sources may carry undetectable contaminants. Therefore, the current trend tends to reduce or even eliminate the usage of the materials derived from animal sources to reduce the risk of contamination.

Moreover, among the commercially available cell carrier, except the related products using Alginate as a base material, other synthetic materials all experienced difficulties in controlling the degradation process, causing challenges in recovering the cells. Since the related product using Alginate as a base material requires a high concentration of calcium ion during cell culturing process, damage to the cells or tendency of inducing differentiation of some cells (for example, Mesenchymal Stem Cell) may occur. In addition, during the degradation of Alginate, utilization of calcium ion chelator is required, and high concentration of the calcium ion chelator may cause damage to the cells. The current cell mass production technology still remains at a conventional two dimensional flat plate culture method, and the process cannot be adapted to a larger scale production.

Therefore, a contaminant-free cell culture design for high volume cell expansion and recovery with optimal cell quality has become the problem eager to be solved by researchers.

### SUMMARY OF THE INVENTION

The disclosure provides a cell culture carrier module which can effectively enhance cell recovery rate.

The disclosure provides a cell culture carrier module, which includes at least one cell culture carrier capable of transforming between a two dimensional structure and a three dimensional structure. The cell culture carrier exhibits the two dimensional structure in a loosened state and exhibits the three dimensional structure in a compressed state

The disclosure provides a bioreactor. The bioreactor includes the foregoing cell culture carrier module.

The disclosure provides a cell recovery method, which includes the following steps. A cell culture carrier module is provided, the cell culture carrier module includes at least one cell culture carrier capable of transforming between a two dimensional structure and a three dimensional structure, the cell culture carrier exhibits the two dimensional structure in a loosened state and exhibits the three dimensional structure in a compressed state. Cell culture is performed when the cell culture carrier is in the three dimensional structure state, and cell recovery is performed when the cell culture carrier is in the two dimensional structure state.

Based on the foregoing, the cell culture carrier module of the disclosure includes the cell culture carrier capable of transforming between the two dimensional structure and the three dimensional structure. As such, when cell cultured is performed and the cell culture carrier is in the three dimensional structure state, the three dimensional structure of the cell culture carrier is able to provide more surface area and space for cell growth, thereby enhancing the amount of cells cultured. In addition, when cell recovery is performed and the cell culture carrier is in the two dimensional structure state, the loosened structure is able to allow the cell culture carrier and the cell detachment enzyme to react sufficiently, which facilitates the detachment of the cells that grew on an inner layer of the cell culture carrier, thereby enhancing cell recovery rate.

To make the above features and advantages of the disclosure more comprehensible, several embodiments accompanied with drawings are described in detail as follows.

### BRIEF DESCRIPTION OF THE DRAWINGS

FIG. 1 is a schematic view of a cell culture carrier according to an embodiment of the disclosure.

FIG. 2 is a schematic view of a spiral shaped cell culture carrier according to an embodiment of the disclosure.

FIG. 3 is a schematic view of a randomly organized fiber form cell culture carrier according to an embodiment of the disclosure.

FIG. 4 is a schematic view of a cell culture carrier according to another embodiment of the disclosure.

FIG. 5a is a schematic view of a cell culture carrier according to a first embodiment of the disclosure.

FIG. 5b is a schematic view of the cell culture carrier of FIG. 5a after being compressed.

FIG. 6a is a schematic view of a cell culture carrier according to a second embodiment of the disclosure.

FIG. 6b is a schematic view of the cell culture carrier of FIG. 6a after being compressed.

FIG. 7a is a schematic view of a cell culture carrier according to a third embodiment of the disclosure.

FIG. 7b is a schematic view of the cell culture carrier of FIG. 7a after being compressed.

FIG. 8 is a flow chart illustrating steps for a cell recovery process according to an embodiment of the disclosure.

FIG. 9 is a schematic view of a bioreactor according to an embodiment of the disclosure.

FIG. 10 is a growth curve of an African green monkey kidney cell line (VERO) cultured in the cell culture carrier of the disclosure after 21 days.

FIG. 11 is a growth curve of a Human Adipose-Derived Stem Cell (ADSC) cultured in the cell culture carrier of the disclosure after 21 days.

### DESCRIPTION OF THE EMBODIMENTS

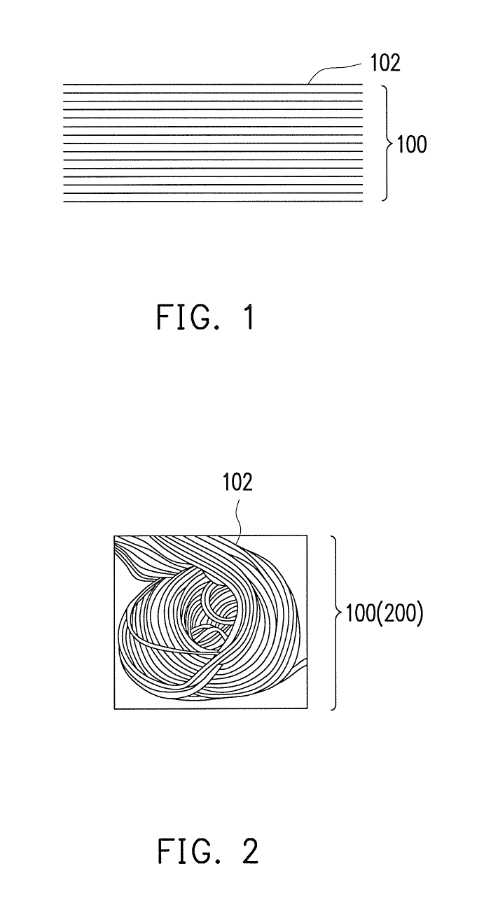

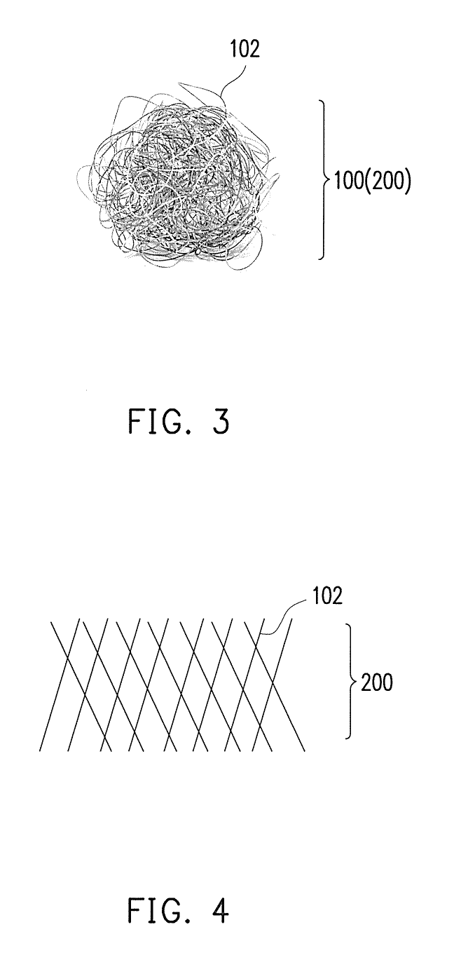

FIG. 1 is a schematic view of a cell culture carrier according to an embodiment of the disclosure. FIG. 2 is a schematic view of a spiral shaped cell culture carrier according to an embodiment of the disclosure. FIG. 3 is a schematic view of a randomly organized fiber form cell culture carrier according to an embodiment of the disclosure.

Referring to FIG. 1 to FIG. 3, the cell culture carrier module includes a cell culture carrier 100 capable of transforming between a two dimensional structure and a three dimensional structure. The cell culture carrier 100 exhibits the two dimensional structure (referring to FIG. 1) in a loosened state and exhibits the three dimensional structure (referring to FIG. 2 and FIG. 3) in a compressed state. In the embodiment of FIG. 1, the two dimensional structure is exemplified by a parallel line array, but it construes no limitation in the disclosure. The three dimensional structure is, for example, spiral shaped (FIG. 2) or randomly organized fiber form (FIG. 3).

For instance, the cell culture carrier 100 includes a plurality of cell culture threads 102. As illustrated in FIG. 1, the plurality of cell culture threads 102 are arranged in a parallel manner to form the parallel line array exhibiting the two dimensional structure. A material of the cell culture carrier 100 is, for example, Polyester (PET), Nylon, Polyethylene (PE), Polypropylene (PP), Polyvinyl Chloride (PVC), Polystyrene (PS), Ethylene Vinyl Acetate (EVA), polyurethane (PU), or the like. However, they construe no limitation in the disclosure. Materials having fiber drawing properties (For example, striped sheets or threadlike sheets) are all able to be adapted as the material for the cell culture carrier of the disclosure.

The cell culture carrier 100 may be a cell adhesion material or a material having cell adhesion property after processing. The foregoing processing method includes surface modification, surface coating, surface microstructurization, or the like. Surface modification, for example, can be achieved by performing plasma modification on a surface of the cell adhesion material or the cell non-adhesion material to render the surface encompassing cell adhesion property, thereby facilitating the attachment of the cells. Surface coating includes coating collagen, chitosan, gelatin, Alginate, or the like onto the surface of the cell adhesion material or the cell non-adhesion material to facilitate the attachment of the cells, but they construe no limitation in the disclosure. Surface microstructurization, for example, can be achieved by performing laser cutting on the surface of the cell adhesion material or the cell non-adhesion material to form microchannels, thereby facilitating the attachment of the cells. However, the processing methods of the disclosure are not limited thereto, other processing method able to enhance the cell attachment property can also be adapted in the disclosure.

In the present embodiment, the method of transforming the cell culture carrier 100 from the two dimensional structure to the third dimensional structure is, for example, by squeezing or twisting the cell culture carrier 100 in the two dimensional structure state such that the cell culture carrier 100 exhibiting the two dimensional structure is transformed to the three dimensional structure state through compression. The three dimensional structure is, for example, spiral shaped (FIG. 2) or randomly organized fiber form (FIG. 3). For instance, twisting or compressing the cell culture carrier 100 exhibiting the two dimensional structure can be achieved by hand, but it construes no limitation in the disclosure. In another embodiment, auxiliary tools may be adapted to twist or compress the cell culture carrier 100 exhibiting the two dimensional structure.

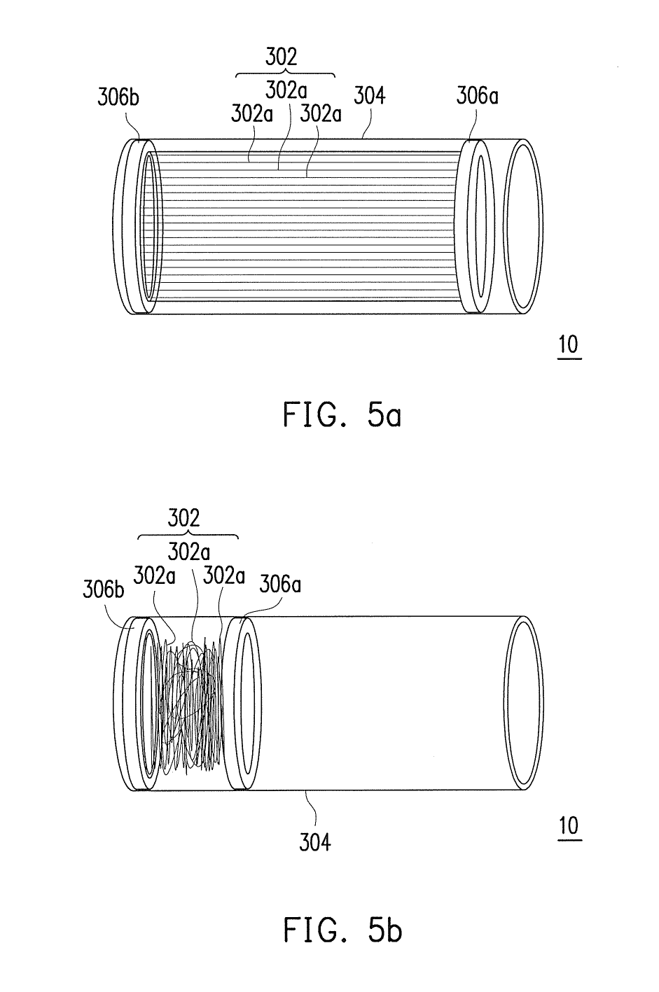

The method of transforming the cell culture carrier 100 from the three dimensional structure to the two dimensional structure is, for example, by loosening or straightening the cell culture carrier 100 in the three dimensional structure state such that the cell culture carrier 100 exhibiting the three dimensional structure is transformed to the two dimensional structure state. For instance, the loosening or straightening of the cell culture carrier 100 exhibiting the three dimensional structure can be achieved by hand, but it construes no limitation in the disclosure. In another embodiment, auxiliary tools (referring to FIG. 5a to FIG. 7b) may be adapted to loosen or straighten the cell culture carrier 100 exhibiting the three dimensional structure.

In the foregoing embodiment, since the cell culture carrier module includes the cell culture carrier 100 capable of transforming between the two dimensional structure and the three dimensional structure, when cell culture is performed and the cell culture carrier 100 is in the three dimensional structure state, the three dimensional structure of the cell culture carrier 100 is able to provide more surface area and space for cell growth, thereby enhancing the amount of cells cultured. In addition, when cell recovery is performed and the cell culture carrier 100 is in the two dimensional structure state, the loosened structure is able to allow the cell culture carrier 100 and the cell detachment enzyme to react sufficiently, which facilitates the detachment of the cells that grew on an inner layer of the cell culture carrier 100, thereby enhancing cell recovery rate.

FIG. 4 is a schematic view of a cell culture carrier according to another embodiment of the disclosure.

Referring to FIG. 4, a cell culture carrier 200 can also transform between a two dimensional structure and a three dimensional structure. The cell culture carrier 200 exhibits the two dimensional structure (referring to FIG. 4) in a loosened state and exhibits the three dimensional structure (referring to FIG. 2 and FIG. 3) in a compressed state. Referring to FIGS. 1 and 4 simultaneously, the difference between the cell culture carrier 200 of FIG. 4 and the cell culture carrier 100 of FIG. 1 lies in: the two dimensional structure of the cell culture carrier 200 is a crossed line array. Namely, cell culture threads 102 can arrange in a crossed manner to foal′ the crossed line array exhibiting the two dimensional structure. Moreover, the same component within the cell culture carrier 200 and the cell culture carrier 100 are denoted by the same reference numeral and the descriptions thereof are omitted.

FIG. 5a is a schematic view of a cell culture carrier according to a first embodiment of the disclosure. FIG. 5b is a schematic view of the cell culture carrier of FIG. 5a after being compressed.

Referring to FIG. 5a and FIG. 5b simultaneously, the cell culture carrier module 10 includes a cell culture carrier 302, an external sleeve 304, and two fixing components 306a, 306b. The cell culture carrier 302 is, for example, at least one of the cell culture carriers 100, 200 according to the foregoing embodiments. The cell culture carrier 302 may includes a plurality of cell culture threads 302a.

The fixing components 306a, 306b are respectively disposed at both ends of the cell culture carrier 302. The cell culture carrier 302 is located in the external sleeve 304 and is fixed onto the fixing components 306a, 306b. A material of the external sleeve 304 and fixing components 306a, 306b are, for example, Polyester (PET), Nylon, Polyethylene (PE), Polypropylene (PP), Polyvinyl Chloride (PVC), Polystyrene (PS), Ethylene Vinyl Acetate (EVA), polyurethane (PU), Polycarbonate (PC), glass, or the like, but they construe no limitation in the disclosure.

In the present embodiment, the cell culture carrier 302 is transformed from the two dimensional structure to the three dimensional structure (as illustrated in FIG. 5b) by pushing the fixing component 306a into the external sleeve 304 to compress the cell culture carrier 302. Similarly, the cell culture carrier 302 is transformed from the three dimensional structure to the two dimensional structure (as illustrated in FIG. 5a) by withdrawing the fixing component 306a from the external sleeve 304.

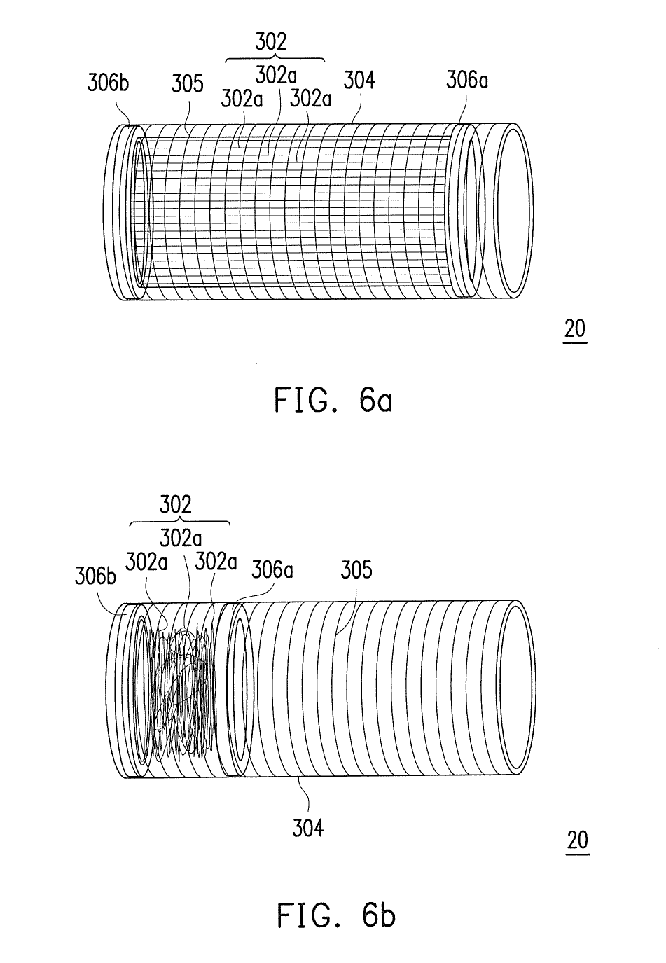

FIG. 6a is a schematic view of a cell culture carrier according to a second embodiment of the disclosure. FIG. 6b is a schematic view of the cell culture carrier of FIG. 6a after being compressed.

A cell culture carrier module 20 of FIG. 6a and FIG. 6b is similar to the cell culture carrier module 10 of FIG. 5a and FIG. 5b. In FIG. 6a and FIG. 6b, the same element as that of FIG. 5a and FIG. 5b are denoted by the same reference numeral and the descriptions thereof are omitted. Referring to FIG. 6a and FIG. 6b simultaneously, the difference between the cell culture carrier module 20 of FIG. 6a and FIG. 6b and the cell culture carrier module 10 of FIG. 5a and FIG. 5b lies in: in the present embodiment, an inner wall of the external sleeve 304 has a screw thread 305.

In the present embodiment, the cell culture carrier 302 is being twisted to exhibits a spiral manner due to compression by screwing the fixing component 306a along the screw thread 305 into the external sleeve 304, and the cell culture carrier 302 is transformed from the two dimensional structure to the three dimensional structure (as illustrated in FIG. 6b). Similarly, the cell culture carrier 302 is loosened by unscrewing the fixing component 306a along the screw thread 305 from the external sleeve 304, and the cell culture carrier 302 is transformed from the three dimensional structure to the two dimensional structure (as illustrated in FIG. 6a).

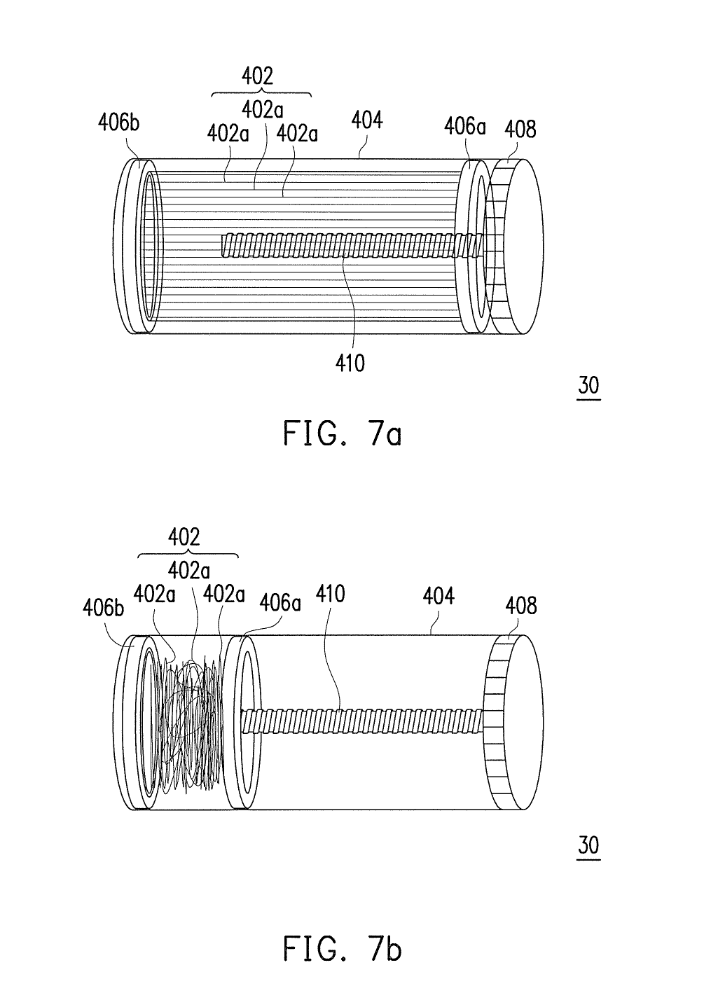

FIG. 7a is a schematic view of a cell culture carrier according to a third embodiment of the disclosure. FIG. 7b is a schematic view of the cell culture carrier of FIG. 7a after being compressed.

Referring to FIG. 7a and FIG. 7b simultaneously, the cell culture carrier module 30 of the present embodiment includes a cell culture carrier 402, an external sleeve 404, two fixing components 406a, 406b, a driving element 408, and a screw 410. The cell culture carrier 402 is, for example, at least one of cell culture carriers 100, 200 according to the foregoing embodiments. The cell culture carrier 402 may includes a plurality of cell culture threads 402a.

The fixing components 406a, 406b are respectively disposed at both ends of the cell culture carrier 402. The cell culture carrier 402 is arranged linearly in the external sleeve 404 and is fixed onto the fixing components 406a, 406b. A material of the external sleeve 404 and fixing components 406a, 406b are, for example, Polyester (PET), Nylon, Polyethylene (PE), Polypropylene (PP), Polyvinyl Chloride (PVC), Polystyrene (PS), Ethylene Vinyl Acetate (EVA), polyurethane (PU), Polycarbonate (PC), glass, or the like, but they construe no limitation in the disclosure.

The driving element 408 is disposed at an end of the external 404 which is close to the fixing component 406a. The screw 410 is connected to the driving element 408 and penetrated through the fixing component 406a. Therefore, the fixing component 406a is pushed into the external sleeve 404 by driving the screw 410 through the driving element 408, and the cell culture carrier 402 is transformed from the two dimensional structure to the three dimensional structure (as illustrated in FIG. 7b). Similarly, the fixing component 406a is withdrawn from the external sleeve 404 by driving the screw 410 through the driving element 408, and the cell culture carrier 402 is transformed from the three dimensional structure to the two dimensional structure (as illustrated in FIG. 7a).

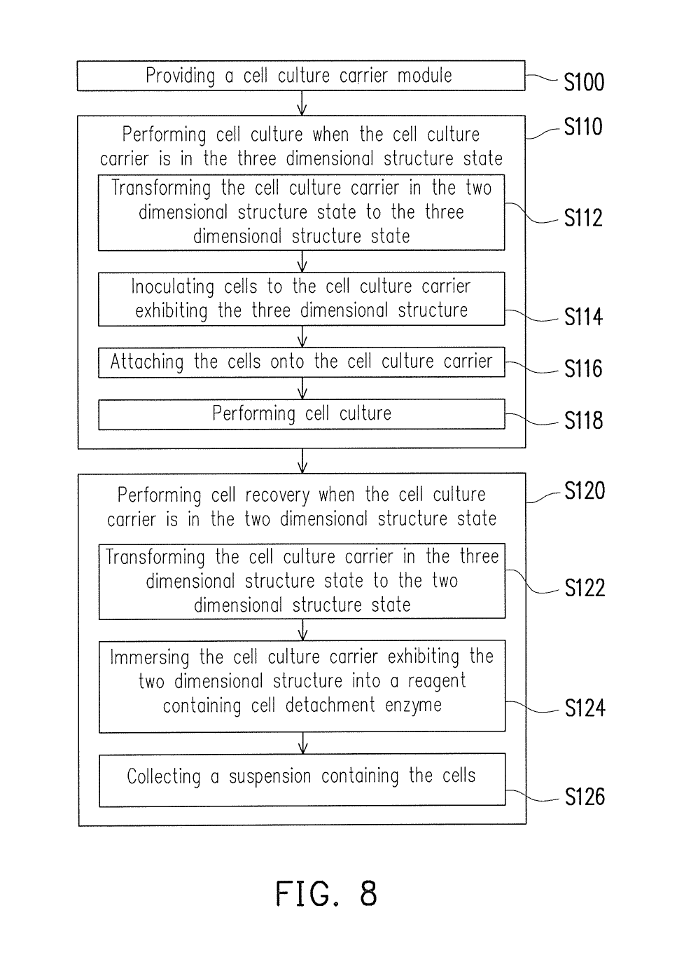

FIG. 8 is a flow chart illustrating steps for a cell recovery process according to an embodiment of the disclosure.

Referring to FIG. 8, the detailed description of the steps for performing the cell recovery are presented below:

First, step S100 is performed: providing a cell culture carrier module. The cell culture carrier module may be at least one of the cell culture carrier modules of FIG. 1 to FIG. 7b. The foregoing cell culture carrier module includes a cell culture carrier capable of transforming between a two dimensional structure and a three dimensional structure. The cell culture carrier exhibits the two dimensional structure in a loosened state and exhibits the three dimensional structure in a compressed state.

Next, step S110 is performed: performing cell culture when the cell culture carrier is in the three dimensional structure state. Step 110 may further includes sub-steps S112, S114, S116, and S118. Sub-step S112 is the step of transforming the cell culture carrier in the two dimensional structure state to the three dimensional structure state. The method of transforming the cell culture carrier from the two dimensional structure state to the three dimensional structure state has been described in detail in the foregoing embodiments, thus the descriptions thereof are omitted herein.

Subsequently, sub-step S114 is performed: inoculating cells to the cell culture carrier exhibiting the three dimensional structure. The cultured cells are, for example, stem cells or cells subject to differentiation, but they construe no limitation to the disclosure. Specifically, the cultured cells are, for example, African green monkey kidney cell line (VERO), Human Adipose-Derived Stem Cell (ADSC), Mesenchymal Stem Cell (MSC), Madin-Darby Canine Kidney (MDCK) cell, Human Embryonic Kidney 293 (HEK 293) cell, or the like, but they construe no limitation to the disclosure. In the present embodiment, a culture medium is first added into the cell culture carrier such that the entire cell culture carrier exhibiting the three dimensional structure is full of the culture medium, and then the cells are inoculated into the cell culture carrier. In another embodiment, the cell culture medium containing cells may be uniformly added into the cell culture carrier exhibiting the three dimensional structure directly such that the entire cell culture carrier exhibiting the three dimensional structure is full of the culture medium. The culture medium is standard growth culture medium for cell culture used conventionally, and the examples are the culture medium having Fetal Bovine Serum (FBS) or serum free medium (SFM), but they construe no limitation in the disclosure. Moreover, it should be understood that the requirements of the operating concentration of the cell culture medium are also different based on the different cell properties. Therefore, the operating concentration may be adjusted based on cell properties. In addition, growth factors, antibiotics, or the like may be added to the culture medium based on need, as known to persons having ordinary skill in the art.

Next, sub-step S116 is performed: attaching the cells onto the cell culture carrier. In the present embodiment, the cell culture carrier is disposed in an incubator under a specific growth condition (for example, specific temperature, humidity, or carbon dioxide concentration) such that the cells are attached onto the cell culture carrier.

Subsequently, sub-step S118 is performed: performing cell culture. The method of cell culture is, for example, placing the cell culture carrier in the incubator for static culture or dynamic culture. Dynamic culture may be achieved by disturbing the culture medium surrounding the cell culture carrier. The method for disturbing the culture medium is, for example, placing the culture flask having the cell culture carrier module on a magnetic rotatable plate, and the magnetic rotatable plate is able to drive the rotation of the magnet for disturbing the culture medium. In an embodiment, the cell growth number may increase to 100 times or more after cell culture. In another embodiment, the cell growth number may increase to 2000 times or more after cell culture.

It should be noted that since different cells have different properties, the cell culture conditions may be adjusted based on different cell types. For example, when culturing mammalian cells, the cells may be cultured at a condition of 37° C. and 5% of CO₂, while the pH value of the culture medium is maintained within the physiological range. For instance, for most of the animal cells, the suitable pH value of the culture medium is 7.2 to 7.4.

As compared to the cell culture carrier exhibiting the two dimensional structure, in the present embodiment, since the cell inoculation and the cell culture are performed on the cell culture carrier exhibiting the three dimensional structure, the three dimensional structure of the cell culture carrier is able to provide more surface area and space for cell growth, thereby enhancing the amount of cells cultured. Moreover, in the present embodiment, the cell inoculation and the subsequent cell culture are performed on the cell culture carrier in the compressed state, thus the amount of culture medium being utilized may be reduced, thereby reducing cost.

Next, step S120 is performed: performing cell recovery when the cell culture carrier is in the two dimensional structure state. Step S120 may further includes the following sub-steps S122, S124, and S126.

First, sub-step S122 is performed: transforming the cell culture carrier in the three dimensional structure state to the two dimensional structure state. The method of transforming the cell culture carrier in the three dimensional structure state to the two dimensional structure state has been described in detail in the foregoing embodiments, thus the descriptions thereof are omitted herein.

Subsequently, sub-step S124 is performed: immersing the cell culture carrier exhibiting the two dimensional structure into a reagent containing cell detachment enzyme such that the cells are detached from the cell culture carrier. In an embodiment, the reagent containing cell detachment enzyme may be drop-added in the two dimensional structure state such that the cell culture carrier exhibiting the two dimensional structure is immersed in the reagent containing cell detachment enzyme. In another embodiment, the reagent containing cell detachment enzyme may be drop-added in the three dimensional structure state or during the process of transforming from the three dimensional structure to the two dimensional structure such that the cell culture carrier may be immersed in the reagent containing cell detachment enzyme in the three dimensional state. The cell detachment enzyme is, for example, Trypsin, Tryp LE, Accutase, Accumax, or Collagenase, but they construe no limitation in the disclosure. Other enzyme or reagent capable of cell detachment may also be used.

Next, sub-step S126 is performed: collecting a suspension containing the cells to complete cell recovery.

In the foregoing embodiments, the cell recovery is exemplified when the cell culture carrier is in the two dimensional structure state. In another embodiment, under the condition when the reagent containing cell detachment enzyme is drop-added in the three dimensional structure state or during the process of transforming from the three dimensional structure to the two dimensional structure, other than recovering the cells when the cell culture carrier is in the two dimensional state, the cell recovery may also be performed in the three dimensional structure state or during the process of transforming from the three dimensional structure to the two dimensional structure.

In the present embodiment, since the cell recovery is performed when the cell culture carrier is in the two dimensional structure state, the loosened structure is able to allow the cell culture carrier and the reagent containing cell detachment enzyme to react sufficiently, and the loosened structure is advantageous to the detachment of the cells that grew on an inner layer of the cell culture carrier, thereby enhancing cell recovery rate.

In an embodiment, at least one of the cell culture carrier modules of FIG. 1 to FIG. 7b may be used in a bioreactor to enhance the cell amount and cell recovery rate of the bioreactor.

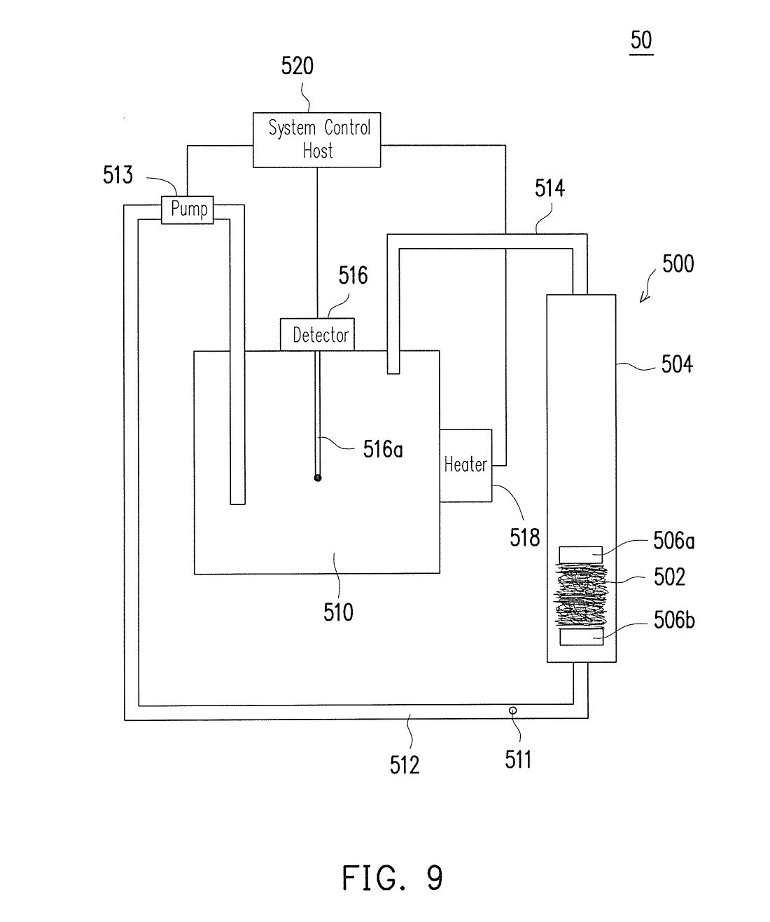

FIG. 9 is a schematic view of a bioreactor according to an embodiment of the disclosure.

Referring to FIG. 9, a bioreactor 50 of the present embodiment includes a cell culture carrier module 500 and a culture medium tank 510. The cell culture carrier module 500 includes a cell culture carrier 502, an external sleeve 504, and two fixing components 506a, 506b. The fixing components 506a, 506b are respectively disposed at both ends of the cell culture carrier 502. The cell culture carrier 502 is located in the external sleeve 504 and is fixed onto the fixing components 506a, 506b.

The culture medium tank 510 includes a culture medium inlet pipe 512 and a culture medium outlet pipe 514. The culture medium tank 510 is connected to both ends of the external sleeve 504 respectively through the culture medium inlet pipe 512 and the culture medium outlet pipe 514. The culture medium inlet pipe 512 may inject the culture medium in the culture medium tank 510 into the external sleeve 504 through a pump 513, and the culture medium outlet pipe 514 may output the culture medium in the external sleeve 504 to the culture medium tank 510. The culture medium inlet pipe 512 includes cell injection holes 511. The cell culture medium containing cells may be injected into the culture medium inlet pipe 512 through the cell injection holes 511 and enters the external sleeve 504 through the culture medium inlet pipe 512.

In the present embodiment, the culture medium tank 510 further includes at least one detector 516 and a heater 518. The detector 516 is disposed on the culture medium tank 510. The detector 516 includes a probe 516, and an end of the probe 516a extends into the culture medium in the culture medium tank 510. The detector 516 tests the culture medium through the probe 516a. The detector 516 is, for example, pH meter, thermometer, or dissolved oxygen meter.

The heater 518 is disposed outside of the culture medium tank 510. The temperature of the culture medium in the culture medium tank 510 may be elevated through the heater 518 such that the culture medium is maintained at a suitable temperature.

Moreover, the bioreactor 50 may further include a system control host 520 which is connected to the pump 513, the detector 516, and the heater 518 to control the input and the output of the culture medium, the detector, and the heater 518.

The steps of the cell recovery process utilizing the foregoing bioreactor will be presented below.

First, the cell culture medium containing cells is injected into the culture medium inlet pipe 512 through the cell injection holes 511. The injected cell culture medium enters the external sleeve 504 through the cell culture inlet pipe 512 and is being inoculated on the cell culture carrier 502 exhibiting the three dimensional structure. In the present embodiment, the volume of the cell culture medium injected into the external sleeve 504 is approximately equal to the volume covering the entire cell culture carrier 502. The cells are allowed to be attached onto the cell culture carrier 502 (approximately 4 to 6 hours, and the duration may be adjusted based on different types of cells).

Subsequently, the culture medium in the culture medium tank 510 is injected into the external sleeve 504 through the culture medium inlet pipe 512, and at the same time, the culture medium in the external sleeve 504 is outputted to the culture medium tank 510 through the culture medium outlet pipe 514, thereby performing the perfusion and circulation of the culture medium.

In order for the culture medium in the culture medium tank 510 to mix uniformly, the mixing of the culture medium in the culture medium tank 510 may be achieved by disturbing the culture medium in the culture medium tank 510 or by oscillating the culture medium tank 510. The method of disturbing the culture medium is, for example, placing the culture medium tank 510 on a magnetic rotatable plate, and the magnetic rotatable plate is able to drive the rotation of the magnet for disturbing the culture medium. The method of oscillating the culture medium tank 510 is, for example, placing the culture medium tank 510 on an oscillator and through shaking or oscillating the culture medium tank 510 by the stir bar, the culture medium in the culture medium tank 510 is mixed.

Next, the cell culture is performed when the culture medium is still under circulation. During this period, the system control host 520 controls the input and the output of the culture medium, the detector 516, and the heater 518. For instance, the detector 516 may be controlled to monitor the growth and metabolism condition of the culture medium and the cells.

After the cells have been culture, all of the culture medium in the external sleeve 504 is outputted to the culture medium tank 510 through the culture medium outlet pipe 514. Phosphate buffer saline (PBS) solution is being utilized to repeatedly rinsing the remaining culture medium on the cell culture carrier 502, and subsequently, the PBS solution is removed.

Subsequently, the reagent containing cell detachment enzyme (for example, Trypsin, Tryp LE, Accutase, Accumax, or Collagenase) is drop-added on the cell culture carrier 502 in the three dimensional structure state to allow the detachment of the cells from the cell culture carrier 502. The cell culture carrier 502 in the three dimensional structure state is transformed to the two dimensional structure state to allow the detachment of the cells that grew on an inner layer of the cell culture carrier 502. The method of transforming the cell culture carrier from the three dimensional structure state to the two dimensional structure state has been described in detail in the foregoing embodiments, thus the descriptions thereof are omitted herein. In other embodiments, the reagent containing cell detachment enzyme may be drop-added when the cell culture carrier 502 is in the two dimensional structure state or the reagent containing cell detachment enzyme may be drop-added during the process when the cell culture carrier 502 is transforming from the three dimensional structure state to the two dimensional structure state.

Thereafter, a suspension containing the cells is collected and subsequent centrifugation process or other processes are performed to complete cell recovery.

The examples of the disclosure will be presented below to explain the disclosure in a more specific manner. However, it will be apparent to those skilled in the art that various modifications and variations can be made to the structure of the present disclosure without departing from the scope or spirit of the disclosure. Therefore, the scope of the present disclosure is not limited to the following examples.

### Dynamic Culture Experiment

### Example 1

In Example 1, the cell culture carrier of FIG. 1 is being utilized and the dynamic cell culture experiment has been conducted based on the cell culture steps illustrated in FIG. 8. The cells to be cultured are African green monkey kidney cell line (VERO). The steps of cell culture are the following: a M199 culture medium (containing 5% of FBS) having VERO cells is inoculated onto the cell culture carrier exhibiting the three dimensional structure, and the VERO cell density in the M199 culture medium is 2×10⁴/cm². The dynamic culture is performed on the cell culture carrier implanted with cells under a condition of 37° C. and 5% CO₂ for 21 days. During this period, new culture medium is replaced every 2 to 3 days, and the cell growth is measured at different point in time.

### Example 2

In Example 2, the cell culture carrier of FIG. 1 is being utilized and the dynamic cell culture experiment has been conducted based on the cell culture steps illustrated in FIG. 8. The cells to be cultured are Human Adipose-Derived Stem Cell (ADSC). The steps of cell culture are the following: a serum free medium (SFM) culture medium having ADSC is inoculated onto the cell culture carrier exhibiting the three dimensional structure, and the ADSC cell density in the serum free medium is 1.5×10³/cm². The dynamic culture is performed on the cell culture carrier inoculated with cells under a condition of 37° C. and 5% CO₂ for 21 days. During this period, new culture medium is replaced every 2 to 3 days, and the cell growth is measured at different point in time.

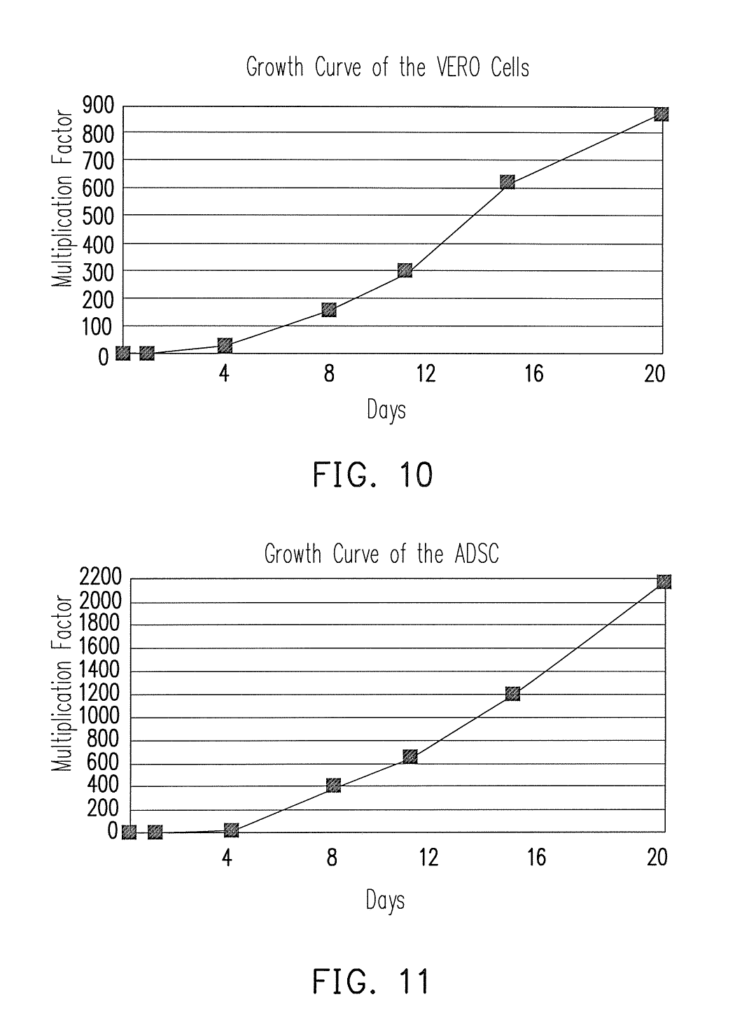

FIG. 10 is a growth curve of an African green monkey kidney cell line (VERO) cultured in the cell culture carrier of the disclosure after 21 days. FIG. 11 is a growth curve of a Human Adipose-Derived Stem Cell (ADSC) cultured in the cell culture carrier of the disclosure after 21 days. As illustrated in FIG. 10 and FIG. 11, the VERO cells of Example 1 and the ADSC of Example 2 both increased along with the number of the culture days. Specifically, after 21 days, the cell growth number of the VERO cell of Example 1 increases to 800 times or more, and after 21 days, the cell growth number of the ADSC of Example 2 increases to 2000 times or more. From the foregoing results, it can be known that the cell culture carrier of the disclosure is not cytotoxic such that the cells may be attached smoothly to render excellent biocompatibility.

### Cell Recovery Rate Test

### Example 3

In Example 3, the cell recovery of the ADSC of Example 2 is conducted based on the cell recovery steps illustrated in FIG. 8, and the cell recovery rate test is performed on the recovered cells. ADSC is being utilized as cells to be cultured. The steps of the cell recovery are the following: the ADSC are cultured respectively in three cell culture carriers exhibiting the three dimensional structure (cell culture carrier A, cell culture carrier B, and cell culture carrier C), and after 10 days of cell culture, each of the cell culture carriers is respectively immersed in Collagenase. Subsequently, each of the cell culture carriers is loosened such that the three dimensional structure is transformed to the two dimensional structure. Continuing to allow each of the cell culture carriers in the two dimensional structure state to be immersed in Collagenase to facilitate the detachment of the cells. Next, the cell amount in the cell suspension and the cell amount remaining on the carrier are calculated (the recovery result for each of the cell culture carriers is listed in detail in the following Table 1).

As shown in Table 1, the cells detached from the cell culture carrier still maintain a survival rate of greater than 80%. In addition, by utilizing the cell culture carrier of the disclosure to perform cell culture, the recovery rate is greater than 80%. From the foregoing results, it can be known that since the cell culture carrier performs cell recovery in the two dimensional structure state in the disclosure, the loosened structure is able to allow the cell culture carrier and Collagenase to react sufficiently, and the loosened two dimensional structure is also able to facilitate the detachment of the cells that grew on an inner layer of the cell culture carrier. As such, the cells unable to detach from the cell culture carrier in the three dimensional structure state may perform detachment under two dimensional structure state, thereby enhancing cell recovery rate.

TABLE 1

| Condition | Survival Rate (%) | Recovery Rate (%) |
|---|---|---|
| Cell Culture Carrier A | 81 | 88 |
| Cell Culture Carrier B | 86 | 93 |
| Cell Culture Carrier C | 87 | 95 |

### Cell Property Test

In order to test the property of the cells cultured by the cell culture carrier of the disclosure, the cell surface of the ADSC recovered based on Example 3 is marked and analyzed through the following operating steps of a flow cytometer.

The operating steps of the flow cytometer are as follows:
1. After the cells are centrifuged, the supernatant medium is removed and suitable volume of MACS Separation Buffer (or 2% of FBS) is added to resuspend the cells such that the concentration of the cell is approximately 1×10⁶/ml to 2×10⁶/ml.
2. Take 100 μl and evenly distribute to each of the test tubes, and the cell amount is 1×10⁵ per tube to 1×10⁶ per tube.
3. Based on the types of the antibody, add suitable amount of antibody under a condition of 2° C. to 8° C. to perform a reaction away from light for 30 minutes.
4. After the addition of 1 ml of Dulbecco's Phosphate Buffered Saline (DPBS), the solution is centrifuged for 5 minutes under the condition of 1500 rpm, and then the supernatant medium is removed.
5. After the addition of 300 μl of DBPS resuspended cells, analysis is performed using the flow cytometer (model: BD FACScan).

In general, the property of ADSC is: high expression in cell markers CD73, CD90, and CD105 of the stem cell, but low expression or no expression in cell markers CD34 and CD45 of the blood cell.

The results are illustrated in Table 2, in which the ADSC cells recovered from cell culture carriers A, B, and C all exhibit a high expression of CD73, CD90, and CD105 and exhibit a low or no expression of CD34 and CD45. The results verified that by using the cell culture carrier of the disclosure to culture and recover ADSC, the stem cell properties thereof still may be maintained.

TABLE 2

| | | | | | CD90 |
|---|---|---|---|---|---|
| Condition | CD45 (%) | CD34 (%) | CD105 (%) | CD73 (%) | (%) |
| Cell Culture | 0.23 | 0.02 | 96.84 | 98.20 | 99.43 |
| Carrier A | | | | | |
| Cell Culture | 1.84 | 0 | 94.72 | 96.68 | 99 |
| Carrier B | | | | | |
| Cell Culture | 0.13 | 0 | 93.04 | 96.83 | 98.35 |
| Carrier C | | | | | |

Based on the foregoing, since the cell culture carrier module of the foregoing embodiments includes the cell culture carrier capable of transforming between the two dimensional structure and the three dimensional structure, not only the cell amount cultured and the cell recovery rate can be enhanced, but also the quality of the proliferated cells can be maintained as well. Namely, the cell can maintains its properties while proliferating, such as the foregoing ADSC recovered still maintains its stem cell properties.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims and their equivalents.

What is claimed is:

1. A cell culture carrier module, comprising:
at least one cell culture carrier capable of transforming between a two dimensional structure and a three dimensional structure, wherein the cell culture carrier exhibits the two dimensional structure in a loosened state and exhibits the three dimensional structure in a compressed state.

2. The cell culture carrier module according to claim 1, wherein the two dimensional structure comprises a parallel line array or a crossed line array.

3. The cell culture carrier module according to claim 1, wherein the three dimensional structure comprises a spiral shape or a coiling shape.

4. The cell culture carrier module according to claim 1, wherein a material of the cell culture carrier comprises a cell adhesion material or a material having cell adhesion property after processing.

5. The cell culture carrier module according to claim 4, wherein the processing method comprises surface modification, surface coating, or surface microstructurization.

6. The cell culture carrier module according to claim 1, further comprising an external sleeve and at least one fixing component, wherein the fixing component is disposed on at least one end of the cell culture carrier, and the cell culture carrier is located in the external sleeve and is fixed onto the fixing component.

7. The cell culture carrier module according to claim 6, wherein
the cell culture carrier is transformed from the two dimensional structure to the three dimensional structure by pushing the fixing component into the external sleeve to compress the cell culture carrier, and
the cell culture carrier is transformed from the three dimensional structure to the two dimensional structure by withdrawing the fixing component from the external sleeve.

8. The cell culture carrier module according to claim 6, wherein
an inner wall of the external sleeve has a screw thread,
the cell culture carrier is transformed from the two dimensional structure to the three dimensional structure by screwing the fixing component along the screw thread into the external sleeve, and
the cell culture carrier is transformed from the three dimensional structure to the two dimensional structure by unscrewing the fixing component along the screw thread from the external sleeve.

9. The cell culture carrier module according to claim 6, further comprising:
a driving element, disposed at one end of the external sleeve; and
a screw, connected to the driving element and penetrated through the fixing component,
the cell culture carrier is transformed from the two dimensional structure to the three dimensional structure by driving the screw through the driving element and pushing the fixing component into the external sleeve, and
the cell culture carrier is transformed from the three dimensional structure to the two dimensional structure by driving the screw through the driving element and withdrawing the fixing component from the external sleeve.

10. A bioreactor, comprising the cell culture carrier module according to claim 1.

11. A cell recovery method, comprising:
providing a cell culture carrier module, wherein the cell culture carrier module comprises at least one cell culture carrier capable of transforming between a two dimensional structure and a three dimensional structure, the cell culture carrier exhibits the two dimensional structure in a loosened state and exhibits the three dimensional structure in a compressed state;
performing cell culture when the cell culture carrier is in a three dimensional structure state; and
performing cell recovery when the cell culture carrier is in a two dimensional structure state.

12. The cell recovery method according to claim 11, wherein the method of transforming the cell culture carrier exhibiting the two dimensional structure to the cell culture carrier exhibiting the three dimensional structure comprises twisting or squeezing the cell culture carrier exhibiting the two dimensional structure.

13. The cell recovery method according to claim 11, wherein the step of performing the cell recovery when the cell culture carrier is in the two dimensional structure state comprises:
immersing the cell culture carrier exhibiting the two dimensional structure into a reagent containing cell detachment enzyme such that cells are detached from the cell culture carrier; and
collecting a suspension containing the cells.

14. The cell recovery method according to claim 13, wherein the cell detachment enzyme comprises Trypsin, Tryp LE, Accutase, Accumax, or Collagenase.

15. The cell recovery method according to claim 11, further comprising adding a reagent containing the cell detachment enzyme to the cell culture carrier in the three dimensional structure state or in the process of transforming the three dimensional structure to the two dimensional structure.

16. The cell recovery method according to claim 15, further comprising performing the cell recovery in the three dimensional structure state or in the process of transforming the three dimensional structure to the two dimensional structure.

17. The cell recovery method according to claim 11, wherein the cell culture carrier module further comprises an external sleeve and at least one fixing component, the fixing component is disposed on at least one end of the cell culture carrier, and the cell culture carrier is located in the external sleeve and is fixed onto the fixing component.

18. The cell recovery method according to claim 17, wherein
the cell culture carrier is transformed from the two dimensional structure to the three dimensional structure by pushing the fixing component into the external sleeve to compress the cell culture carrier, and
the cell culture carrier is transformed from the three dimensional structure to the two dimensional structure by withdrawing the fixing component from the external sleeve.

19. The cell recovery method according to claim 17, wherein
an inner wall of the external sleeve has a screw thread,
the cell culture carrier is transformed from the two dimensional structure to the three dimensional structure by screwing the fixing component along the screw thread into the external sleeve, and
the cell culture carrier is transformed from the three dimensional structure to the two dimensional structure by unscrewing the fixing component along the screw thread from the external sleeve.

20. The cell recovery method according to claim 11, wherein the cell culture carrier module further comprising:
a driving element, disposed at one end of the external sleeve; and
a screw, connected to the driving element and penetrated through the fixing component,
the cell culture carrier is transformed from the two dimensional structure to the three dimensional structure by driving the screw through the driving element and pushing the fixing component into the external sleeve, and
the cell culture carrier is transformed from the three dimensional structure to the two dimensional structure by driving the screw through the driving element and withdrawing the fixing component from the external sleeve.

21. The cell recovery method according to claim 11, wherein a material of the cell culture carrier comprises a cell adhesion material or a material having cell adhesion property after processing.

22. The cell recovery method according to claim 21, wherein the processing method comprises surface modification, surface coating, or surface microstructurization.
